(19) United States
(12) Patent Application Publication
(10) Pub. No.: US 2005/0118576 A1
(43) Pub. Date: Jun. 2, 2005

(54) Novel method and assays for yeast-based drug screening

(76) Inventors: Thomas Meier, Basel (CH); Matthias Jauslin, Basel (CH); Claude A. Nuoffer, Therwill (CH)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/333,639

(22) PCT Filed: Jul. 11, 2001
(86) PCT No.: PCT/EP01/07984

(30) Foreign Application Priority Data
Jul. 20, 2000 (EP) 001155845

(51) Int. Cl. C12Q 1/68; C12Q 1/18; C12N 1/18; C12N 15/74

(52) U.S. Cl. 435/6; 435/32; 435/483; 435/254.2

(57) ABSTRACT

The present invention provides a method wherein yeast mutants deficient in the expression of the yeast homolog of frataxin are applied for the identification and/or evaluation of pharmaceutically active compounds.

The invention concerns especially a method wherein the yeast strain W303-1B/Δydl120w::Kan^{R} is applied for the identification and/or evaluation of chemical and biochemical compounds that protect W303-1B/Δydl120w::Kan^{R} yeast from chemical stress. Furthermore, the invention concerns the application of said method in new cell-based assays to be used for drug screening.

This invention concerns a cell-based method as well as the application of said method in screening assays for the identification and validation of novel drug candidates with special emphasis on yeast-based screening procedures for pharmaceutically active chemical and biochemical compounds.

Said method uses a yeast strain in which the gene encoding the “yeast homolog of frataxin” (see below for specifications) is disrupted. Frataxin is a nuclear encoded protein involved in the regulation of iron homeostasis of mitochondria as for example in yeast, animals and human tissue. A reduced amount of frataxin in humans leads to the development of Friedreich Ataxia, which is the most frequent hereditary ataxia with an estimated disease incidence of 1 in 30,000 Caucasians. Friedreich Ataxia is an autosomal-recessive neurodegenerative disease characterized by progressive gait and limb ataxia, dysarthria, lower limb areflexias, decreased vibration sense, and muscular weakness of the legs. Non-neurological signs include hypertrophic cardiomyopathy and increased incidence of diabetes mellitus. International Patent Application WO97/32996A1 describes the human frataxin gene and its application in molecular diagnosis of Friedreich Ataxia. Onset of symptoms usually occurs around puberty, and typically before the age of 25 years. Life expectancy averages only to 40 to 50 years and there is currently no effective treatment available.

At the cellular and biochemical level, it has been found that frataxin deficiency leads to an accumulation of excess iron in mitochondria of a cell and causes cell damages as a consequence of iron-catalyzed formation of reactive radicals.

Yeast (Saccharomyces cerevisiae) deficient in the YDL120 gene (Genbank, accession numbers Z74168 and NC001136) is considered to be a cellular model for the investigation of the human disease, Friedreich Ataxia. This is based on the observation that YDL120 deficient yeast exhibits several biochemical characteristics that are reminiscent of the pathological manifestation of Friedreich Ataxia as described by Koutnikova, H., Campuzano, V., Foury F., Dollé, P., Cazzalini, O., Koenig M. (Nature Genetics 16, 345-351 (1997)); Foury, F., Cazzalini, O. (FEBS Letters 411, 373-377 (1997)); Babcock, M., de Silva, D., Oaks, R., Davis-Kaplan, S., Jiralerspong, S., Montermini, L., Pandolfo, M., Kaplan, J. (Science 267, 1709-1712 (1997)); Foury, F. (FEBS Letters 456, 281-284 (1999)); Radisky, D. C., Babcock, M. C., Kaplan, J. (J. Biol. Chem. 274, 4497-4499 (1999)).

Most prominent is the accumulation of iron when grown in iron-supplemented medium. Like in affected tissue (e.g. heart muscle) from Friedreich Ataxia patients where significant accumulation of iron in mitochondria can be observed (Delatycki, M. B., Camakaris, J., Brooks, H., Evans-Whipp, T., Thornburn, D. R., Williamson, R., Forrest S. M. (Ann Neurol 45, 673-675 (1999)), also mitochondria of YDL120 deficient yeast accumulate iron to the extent that it can be visualized in the electron microscope (Knight, S. A., Sepuri, N. B. Pain, D., Dancis, A.; J. Biol. Chem. 273, 18389-18393 (1998)). Depending on the culture condition, an up to 10-15 fold increase in mitochondrial iron accumulation has been reported. It is believed that accumulation of intramitochondrial iron results in oxidative stress due to the iron-catalyzed production of reactive radicals. This is shown by the finding that growth of YDL120 deficient yeast is also impaired when cultured in the presence of pro-oxidant molecules (e.g. hydrogen peroxide) as described in the above mentioned references. As a consequence of oxidative damage to the mitochondrial genome YDL120 deficient yeast exhibits:
1. increased frequency in the partial or complete loss of mitochondrial DNA leading to the formation of so called rho⁻ mutants which are unable to perform oxidative phosphorylation.
2. reduced growth rates when cultured in medium containing non-fermentable carbon sources, such as alcohols like ethanol, glycerol, or raffinose.
3. impaired enzymatic activities of cytosolic and mitochondrial enzymes that contain iron-sulfur clusters (e.g. aconitase). Such enzymes are susceptible to oxidative damages and have been shown to exhibit lower enzymatic activities also in human patient tissue samples (Rötig, A., de Lonlay, P., Chretien, D., Foury, F., Koenig, M., Sidi, D., Munnich, A., Rustin, A. (Nature Genetics 17, 215-217 (1997)).

Surprisingly it has now been found that YDL120 deficient yeast can be employed in cell-based assays for the identification, and/or evaluation of chemical and biochemical compounds that protect YDL120 deficient yeast from experimentally imposed cellular stress. It is anticipated that those compounds also have potential therapeutic activity for the treatment of certain human diseases, in particular disease where mitochondrial malfunction, or any form of cellular damage caused by reactive radicals is involved. In case of Friedreich Ataxia this is based on the aforementioned results that show that inactivation of the YDL120 gene in yeast reduces the mitochondrial respiratory performance in a way that is comparable to mitochondrial damage seen in pathologically affected tissues of human patients. Taken together, the avoidance of cellular stress situations in YDL120 deficient yeast can be seen in a figurative sense comparable to the avoidance of cellular stress in Friedreich Ataxia and other diseases listed thereafter.

The present invention provides for a novel method and a novel cell-based assay system for the identification and evaluation of compounds with pharmacological activities. The assay system relies on the application of yeast mutants deficient in the expression of the “yeast frataxin homolog” (yfh1). Further, the invention relies on the application of yeast strains in which the YDL120 gene sequences are interrupted, or replaced or deleted. In one particular embodiment of the invention the YDL120 gene of the W303-1B parental yeast strain was replaced by gene sequences that render the resulting yeast strain resistant to Kanamycin. The relevant genotype of this yeast strain is:
W303-1B/ΔYDL120(Mat alpha ura3 ade2 his3 trp1 leu2 yfh1ΔKan^{R})
In the following, this particular YDL120 deficient yeast is named W303-1B/Δydl120w::Kan^{R} or “mutant” yeast.
For comparison the following parental strain is used in this invention:
W303-1B (Mat alpha ura3 ade2 his3 trp1 leu2)

This parental strain is referred to as W303-1B yeast or “wild-type” yeast thereafter. Both mutant and parental strains are described in Foury, F., Cazzalini, O. (FEBS Letters 411, 373-377 (1997)). This invention is not restricted to the application of this particular parental strain W303-1B but applies also to other parental yeast strains, such as D273UK or CENPK2 as described in Foury, F., Cazzalini, O. (FEBS Letters 411, 373-377 (1997)) or DY150 as described in Babcock, M., de Silva, D., Oaks, R., Davis-Kaplan, S., Jiralerspong, S., Montermini, L., Pandolfo, M., Kaplan, J. (Science 267, 1709-1712 (1997)). The corresponding frataxin deficient yeast strains are D273UKΔYDL120 or CNPK2 ΔYDL120 or DY150 ΔYDL120, respectively.

The drug-screening method as well as assays using this method described here are suitable for the screening of a broad collection of chemical compounds consisting of molecules as for example derived from synthetic chemistry or combinatorial chemistry, as well as selections of natural compounds in form of purified small molecules or in form of crude extracts. In addition, compounds consisting of amino acids such as peptides, or proteins can be used. In general, compounds can be applied as isolated compounds or as mixtures.

The invention provides for the application of said W303-1B/Δydl120w::Kan^{R} yeast mutant and the corresponding wild-type strain in culture devices and especially in microtiter plates (e.g. 96-well microtiter plate format) using supplemented culture medium. The medium may be applied in liquid or solid form and is supplemented with metal ions (e.g. copper and iron ions) or pro-oxidant molecules as physiological challenge for the growth rate of the W303-1B/Δydl120w::Kan^{R} yeast.

In a specific embodiment of this invention the metal ions may be applied as inorganic copper or iron salts or in form of iron bound for example to transferrin or any other carrier. Pro-oxidant molecules may be of chemical nature (such as hydrogen peroxide, superoxide radicals, or hydroxyl radicals of any source) or may result from specific enzymatic reactions (such as the xanthine/xanthine oxidase system or the metal ion catalyzed Fenton reaction). In addition, cellular stress for W303-1B/Δydl120w::Kan^{R} yeast may result from the application of non-fermentable carbon source provided in the culture medium, as for example ethanol, or glycerol, or raffinose, or lactate or combinations thereof. Exposure to metal ions, the application of pro-oxidant molecules, and the application of non-fermentable carbon sources or a combination thereof are collectively named “cellular stress” conditions thereafter.

The identification or validation of test compounds with pharmacological activities, such as for example metal chelators or antioxidants, relies on the determination of the growth rate of said W303-1B/Δydl120w::Kan^{R} yeast strain exposed to cellular stress conditions in the presence or absence of chemical compounds to be tested. In one particular embodiment of the invention the growth rate of the W303-1B/Δydl120w::Kan^{R} will be determined by photometry readout of the cell density using any form of optical density (OD) measurement. Compounds with potential pharmacological activity are identified by alterations in the growth rate of the W303-1B/Δydl120w::Kan^{R} strain exposed to cellular stress and compounds to be tested either applied simultaneously or successively. The application of the W303-1B/Δydl120w::Kan^{R} strain exposed to cellular stress but not exposed to any compound serves as internal assay reference condition (lower bound of growth rate). Likewise, the application of the parental W303-1B yeast strain in the presence or absence of cellular stress serves as additional reference values to determine chemical compounds to be tested with pharmacological utilities at the basis of anti-oxidative or metal chelating properties.

For example, the application of the W303-1B/Δydl120w::Kan^{R} yeast strain, or of any other yeast strain deficient for the YDL120 gene, exposed to appropriate cellular stress conditions in miniaturized assay systems allows for the screening of large numbers of chemical compounds and offers the possibility to identify compounds that serve as chelators for iron or copper. Such compounds may have therapeutic effects in human diseases caused by the pathological accumulation of iron or copper in certain tissues of the human body. A non-exclusive list of such diseases comprises Friedreich Ataxia, Thalassemia, Menkes's Disease, and Wilson's Disease. This invention also offers the possibility to identify novel chemical compounds that may serve as antioxidants for therapeutic use. A non-exclusive list of human diseases that could be ameliorated by membrane-permeable antioxidants comprises diseases of the central nervous system (e.g. Parkinson's Disease, Alzheimer's Disease, stroke), as well as neuromuscular diseases and diseases affecting the peripheral nervous system (e.g. Amyotrophic Lateral Sclerosis (ALS), Friedreich Ataxia, various forms of muscular dystrophies). In addition, such antioxidant molecules identified with this novel assay system may be applicable as treatment in conjunction with organ transplantation and for the treatment of reperfusion injury after stroke or cardiovascular complications.

The following examples illustrate the invention.

### EXAMPLE 1

### Intramitochondrial Accumulation of Iron in W303-1B/Δydl120w::Kan^{R} Yeast

Wild-type W303-1B and mutant W303-B/Δydl120w::Kan^{R} yeast were grown on YPGE-plates (3% w/v glycerol, 3% v/v ethanol (96%), 1% w/v yeast extract, 2% w/v bactopeptone, 2% w/v agar in water) and single colonies were picked and cultured in 5 ml of YPD medium (2% w/v glucose, 2% w/v bactopeptone, 1% yeast extract, in water) until the OD₆₀₀ (at a dilution of 1:10 in water) was in the range of 0.4 to 0.8. These precultures were brought to a final OD₆₀₀ of 3.0 with sterilized water. From this 100 microliter were used to inoculate 100 ml of YPD medium. FeSO₄ was freshly prepared in 0.1 N HCl and added to the medium in different concentrations and cultured for 20 hours at 30° C. Mitochondria were prepared from yeast following essentially the protocol described by Glick, B. S., Pon, L. A. (Isolation of highly purified mitochondria from Saccharomyces cerevisiae. In Methods in Enzymol. 260, 213-219; Academic Press; New York). The resulting mitochondrial fraction was resuspended in 0.5 ml buffer containing 0.6 M sorbitol and 20 mM 2-[N-morpholino] ethanesulfonate, potassium salt (pH 6.0). The concentration of mitochondrial iron in W303-1B/Δydl120w::Kan^{R} and wild-type yeast was determined by the bathophenanthroline sulfonate (BPS) method described by Tangeras, A., Flatmark, T., Backstrom, D., Ehrenberg, A. (Mitochondrial iron not bound in heme and iron-sulfur centers. Estimation, compartmentation and redox state. Biochim Biophys Acta 589, 162-175 (1980)). The method relies on the principle that BPS forms colored complexes with Fe(II). Ten microliter of the mitochondrial fraction were mixed with 40 microliter PIPES buffer (5 mM piperazine-N,N′bis[2-ethanesulfonic acid] in water; pH 6.5) and 5 microliter of saturated dithionite solution and incubated for 30 minutes at room temperature. Subsequently 50 microliter of a BPS solution (100 mM bathophenanthroline in water) was added and the formation of the BPS/Fe complex was measured spectrophotometrically using a dual wavelength spectrophotometer with the wavelength pair 540/595 nm. The amount of Fe(II) in the samples was calculated from a standard curve obtained through the addition of known iron concentrations to 50 microliter PIPES buffer in the presence of 5 microliter saturated dithionite solution and correlated to the protein concentration of the mitochondrial samples.

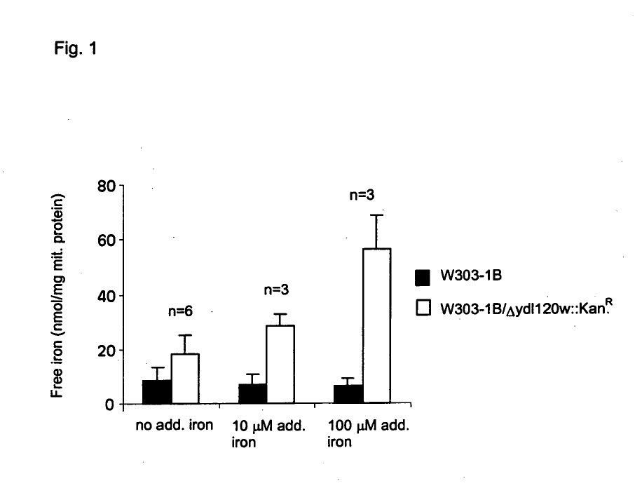

As shown in FIG. 1, the content of mitochondrial non-heme iron in frataxin deficient W303-1B/Δydl120w::Kan^{R} yeast grown in unsupplemented YPD medium was about two-fold increased compared to the iron content of the wild type W303-1B yeast grown under identical conditions. Culture conditions where YPD medium was supplemented with 10 micromolar or 100 micromolar FeSO₄, had no obvious effect on the free mitochondrial iron content of the wild type strain. In contrast, W303-1B/Δydl120w::Kan^{R} yeast accumulated in a dose-dependent way resulting in elevated levels of intramitochondrial iron. Data is mean±standard deviation.

### EXAMPLE 2

### Growth of W303-1B/Δydl120w::Kan^{R} Yeast is Impaired in the Presence of Fe(II)-ions or Cu(II)-ions

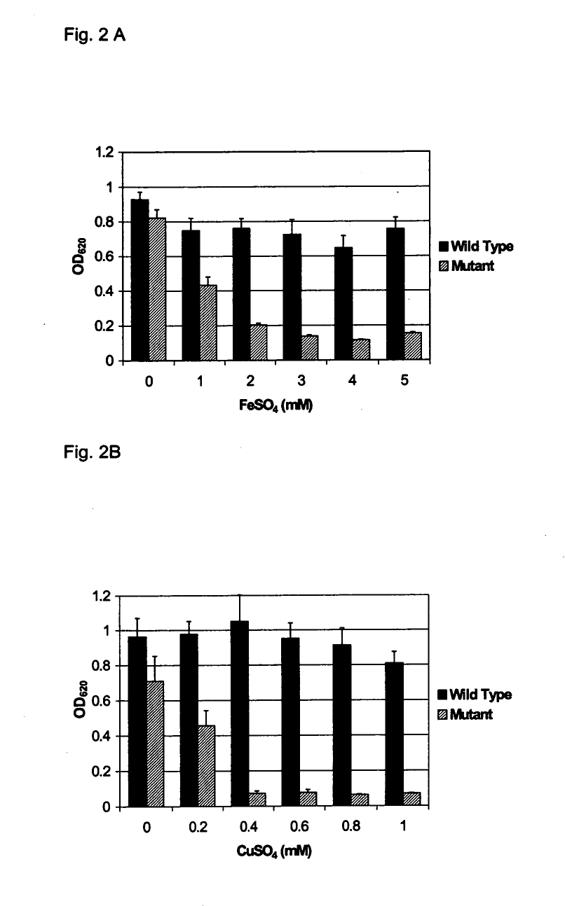

For this experiment, W303-1B/Δydl120w::Kan^{R} and wild-type yeast precultures were grown in YPD medium for 16-24 hours at 30° C. until OD₆₀₀ 0.4-0.6. The precultures were diluted into SD+ medium (6.7 mg/ml yeast nitrogen base w/o amino acids, 2 mg/ml yeast extracts, 0.04 mg/ml adenine sulfate, 0.0625 mg/ml uracil, 0.04 mg/ml L-leucine, 0.03 mg/ml L-histidine-HCl, 0.03 mg/ml L-tryptophane in water) supplemented with variable concentrations of FeSO₄ (dissolved in 0.1 N HCl) or CuSO₄ (dissolved in water). Aliquots of 100 microliter of yeast suspension were dispensed into single wells of a 96-well microtiter plate. In the experiments with FeSO₄ supplementation (FIG. 2A) culture medium for W303-1B/Δydl120w::Kan^{R} and wild-type yeast contained 4% w/v glucose and all wells were adjusted to the same final concentration of HCl and to DMSO (1% v/v) to prevent the solvent from interfering with the results.

FIG. 2A shows the growth of wild-type W303-1B and mutant W303-1B/Δydl120w::Kan^{R} yeast in the presence of FeSO₄ as determined by OD₆₂₀ measurement. Increasing concentrations of FeSO₄ clearly inhibit the growth of the frataxin deficient yeast. Each bar represents the mean and standard deviation of 8 wells in a column of a 96-well microtiter plate. In the experiment with CuSO₄-supplementation (FIG. 2B) culture medium for W303-1B/Δydl120w::Kan^{R} and wild-type yeast contained 2% w/v glucose.

Following 19 hours of culture at 30° C., the growth of yeast was determined by OD₆₂₀ measurement using a microplate reader.

FIG. 2B shows the growth of wild-type W303-1B and mutant W303-1B/Δydl120w::Kan^{R} yeast in the presence of CuSO₄ as determined by OD₆₂₀ measurement. Increasing concentrations of CuSO₄ clearly inhibit the growth of the frataxin deficient yeast. Each bar represents the mean and standard deviation of 8 wells in a column of a 96-well microtiter plate.

### EXAMPLE 3

### Effect of Natural Compounds on the Growth of W303-1B/Δydl120w::Kan^{R} Yeast Cultured in Microtiter Plates in the Presence of 3 mM FeSO₄

Precultures of W303-1B/Δydl120w::Kan^{R} yeast were prepared in YPD-medium as described above. Growth of the preculture was monitored at dilutions of 1:20 in water until OD₆₀₀ of 0.45-0.55 was reached. Precultures were then diluted 1:250 into an appropriate volume of SD+ medium (see above) containing 4% w/v glucose and 3 mM FeSO₄ (diluted into medium from a freshly prepared stock solution of 100 mM FeSO₄ dissolved in 0.1 N HCl). Pure natural compounds were dissolved in DMSO and supplied at concentrations of 3 mg/ml stock solutions. For screening 1 microliter of each of the natural compounds was dispensed in individual wells of a 96-well microtiter plate. To these wells 100 microliter of the FeSO₄ supplemented yeast suspension was added resulting in a final concentration of the pure natural compounds of 0.03mg/ml (=30 ppm). For controls, four wells of each microtiter plate were filled with 100 microliter yeast suspension containing 3 mM FeSO₄ but no natural test-compound (0% growth controls). In addition four wells of each microtiter plate were filled with 100 microliter of yeast suspension without supplemented FeSO₄ (100% growth controls). Where necessary, wells were adjusted to equal concentrations of HCl or DMSO to obtain uniform culture conditions throughout the wells of the microtiter plates. The only variable across the wells of the plate was the presence or absence of FeSO₄ as cell stress and pure natural compounds to be tested for their influence on the growth rate of W303-1B/Δydl120w::Kan^{R} yeast. Microtiter plates were incubated for up to 20 hours at 30° C. in a humid environment. Growth was monitored by measurement of OD₆₂₀ of individual wells in a microplate reader.

The relative growth in each well was calculated according to the following equation:

$${E}_{r} [ \% ] = \frac{V - B}{C - B} \times 100$$

where:
Er=relative growth
V=OD₆₂₀
B=mean of OD₆₂₀ of the 0% growth control (cultures exposed to cellular stress but lacking compounds to be tested)
C=mean of OD₆₂₀ of the 100% growth control (cultures not exposed to cellular stress).

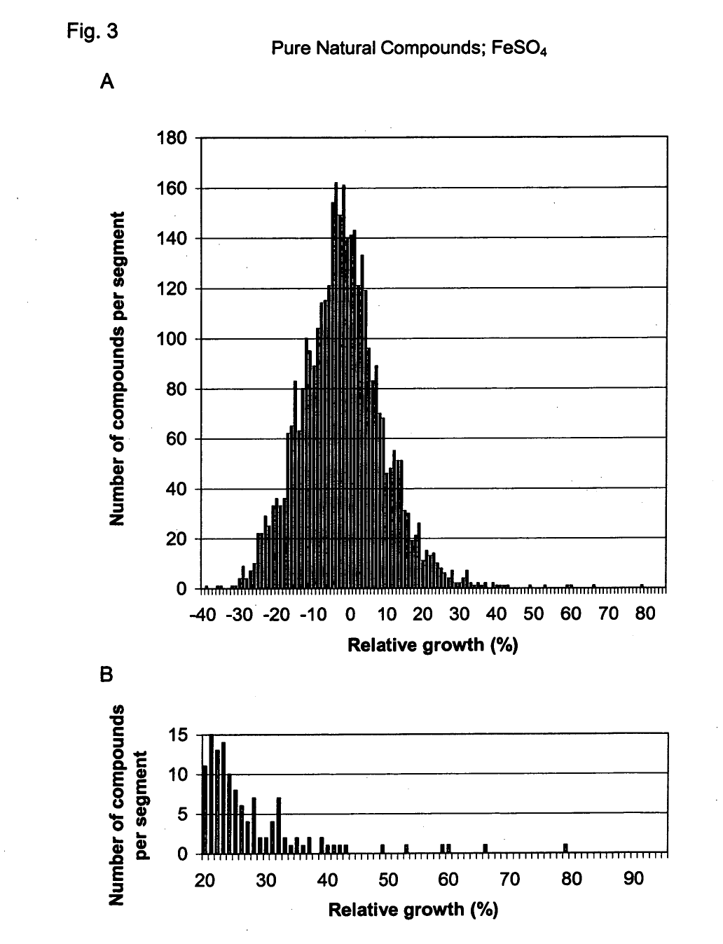

As shown in FIG. 3, in total 1,680 pure natural compounds were analyzed for their effect on the growth of W303-1B/Δydl120w::Kan^{R} yeast in FeSO₄ supplemented medium. Compounds were tested in duplicates at a final concentration of 30 ppm. The frequency distribution of all the relative growth values obtained in this duplicate screen are shown in FIG. 3A (overview) and FIG. 3B (enlarged to show distribution of compounds with positive effect on the growth rates). While the majority of compounds had no influence on the growth rate (main peak centered around 0% relative growth) several compounds induced relative growth of 30% or above.

### EXAMPLE 4

### Effect of Natural Compounds on the Growth of W303-1B/Δydl120w::Kan^{R} Yeast Cultured in Microtiter Plates in the Presence of 0.5 mM CuSO₄

To determine the effect of pure natural compounds on the growth of W303-1B/Δydl120w::Kan^{R} yeast in the presence of CuSO₄ an assay was carried out under essentially the same conditions as described above for the test with FeSO₄ supplemented medium. The following specific changes were undertaken: Instead of FeSO₄, this time precultures of W303-1B/Δydl120w::Kan^{R} yeast were supplemented with 0.5 mM CuSO₄ diluted from a 50 mM stock solution (prepared with water) prior to suspension into individual wells of the microtiter plate. There was no need to adjust for HCl. The identical set of pure natural compounds as described in example 3 was tested for their effect on the growth rate of W303-1B/Δydl120w::Kan^{R} exposed to CuSO₄ and determined by OD₆₂₀ measurement and the calculation described above.

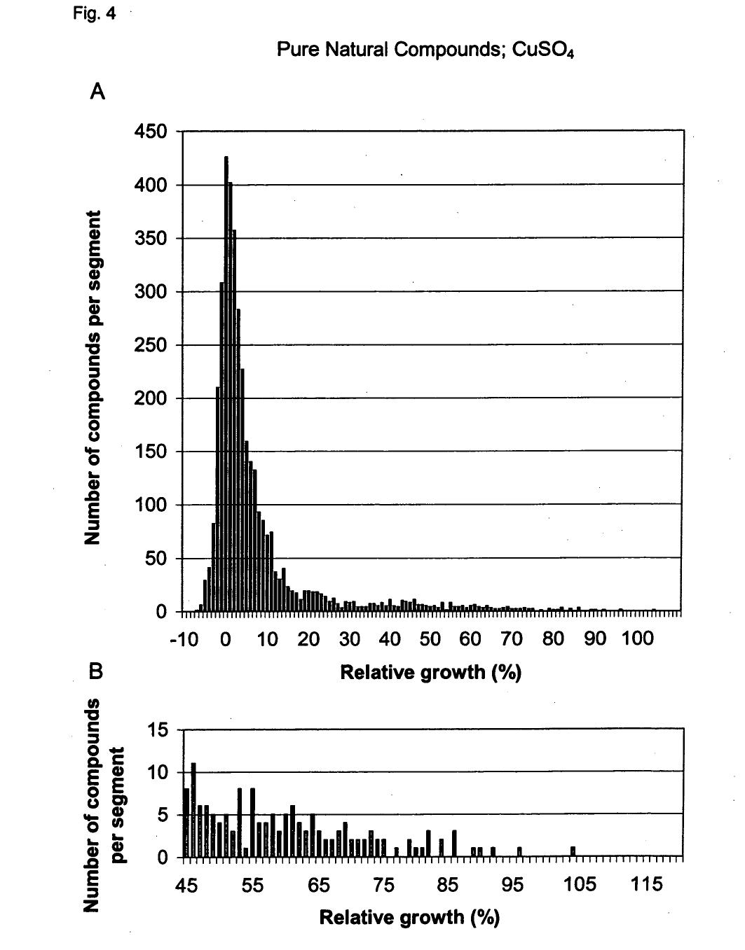

As shown in FIG. 4, in total 1,680 pure natural compounds were analyzed for their effect on the growth of W303-1B/Δydl120w::Kan^{R} yeast in CuSO₄ supplemented medium. Compounds were tested in duplicates at a final concentration of 30 ppm. The frequency distribution of all the relative growth values obtained in this duplicate screen are shown in FIG. 4A (overview) and FIG. 4B (enlarged to show distribution of compounds with positive effect on the growth rates). While the majority of compounds had no influence on the growth rate (main peak centered around 0% relative growth) several compounds induced relative growth of 70% or above.

### EXAMPLE 5

### Effect of Small Molecule Compounds on the Growth of W303-1B/Δydl120w::Kan^{R} Yeast Cultured in Microtiter Plates in the Presence of 3 mM FeSO₄

This assay was carried out essentially as described above (example 3) but this time a selection of small molecules of chemical compounds from a combinatorial chemistry library was used. Again, W303-1B/Δydl120w::Kan^{R} yeast was exposed to 3 mM FeSO₄ as cellular stress and the effect of small molecule compounds on the growth rate was determined by OD₆₂₀ measurement and the calculation described above.

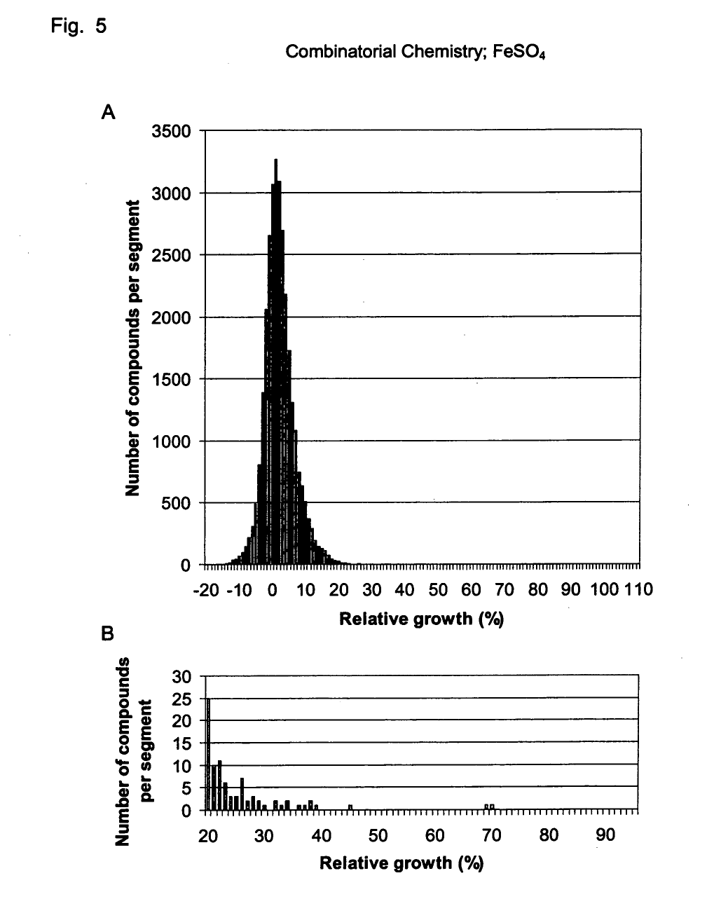

As shown in FIG. 5, in total 15,048 small chemical molecule compounds were analyzed for their effect on the growth of W303-1B/Δydl120w::Kan^{R} yeast in FeSO₄ supplemented medium. Compounds were tested in duplicates at a final concentration of 30 ppm. The frequency distribution of all the relative growth values obtained in this duplicate screen are shown in FIG. 5A (overview) and FIG. 5B (enlarged to show distribution of compounds with positive effect on the growth rates). While the majority of compounds had no influence on the growth rate (main peak centered around 0% relative growth) several compounds induced relative growth of 25% or above.

### EXAMPLE 6

### Effect of Small Molecule Compounds on the Growth of W303-1B/Δydl120w::Kan^{R} Yeast Cultured in Microtiter Plates in the Presence of 0.5 mM CuSO₄

This assay was carried out essentially as described above (example 4) but this time a selection of 15,048 small molecules from a combinatorial chemistry library were used. Again, W303- B/Δydl120w::Kan^{R} yeast was exposed to 0.5 mM CuSO₄ as cellular stress and the effect of small molecule compounds on the growth rate was determined by OD₆₂₀ measurement and the calculation described above.

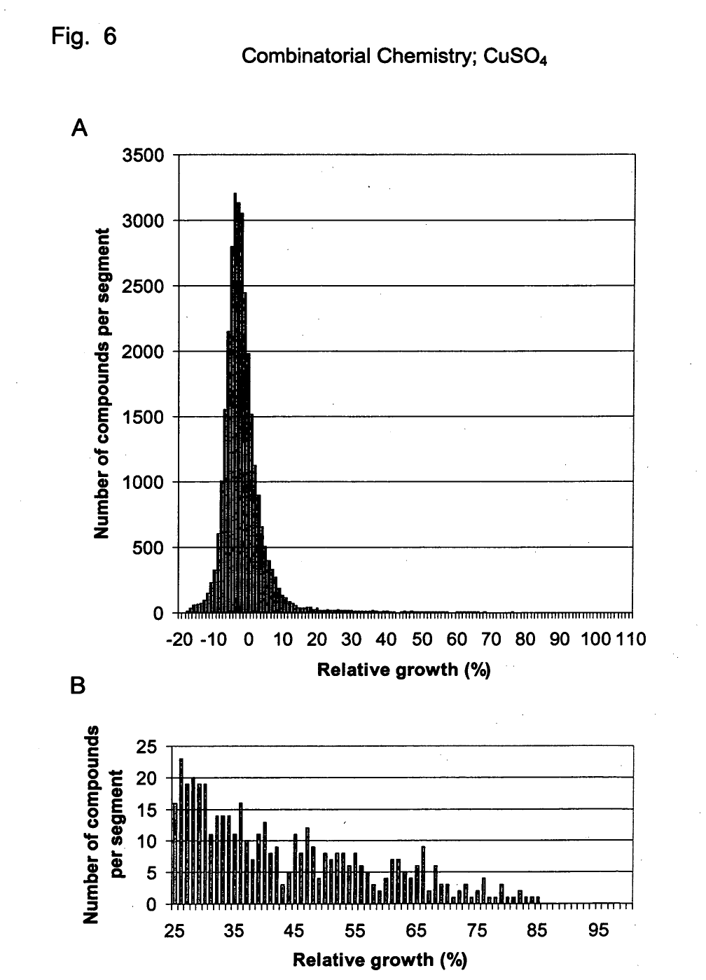

As shown in FIG. 6, in total 15,048 small chemical molecule compounds were analyzed for their effect on the growth of W303-1B/Δydl120w::Kan^{R} yeast in CuSO₄ supplemented medium. Compounds were tested in duplicates at a final concentration of 30 ppm. The frequency distribution of all the relative growth values obtained in this duplicate screen are shown in FIG. 6A (overview) and FIG. 6B (enlarged to show distribution of compounds with positive effect on the growth rates). While the majority of compounds had no influence on the growth rate (main peak centered around 0% relative growth) several compounds induced relative growth of 60% or above.

1. A method of assaying for a compound with pharmacological activity, comprising:
(i) determining a growth rate of a mutant yeast strain deficient in the expression of a yeast frataxin homolog exposed to cellular stress conditions in the presence or absence of a test compound; and
(ii) evaluating the pharmacological activity of said test compound from an alteration in growth rate of the mutant yeast strain in the presence or absence of said test compound.

2. The method of claim 1, wherein said mutant yeast strain includes a disrupted YDL120 gene, and wherein said mutant yeast strain is selected from the group consisting of D273UK, CENPK2, DY150 and W303-1B.

3. The method of claim 2, wherein the mutant yeast strain is W303-1B/Δydl120w: :Kan^{R} and wherein the test compound is pharmaceutically active.

4. The method of claim 2, wherein the mutant yeast strain is W303-1B/Δydl120w::Kan^{R} and wherein the test compound is a chemical or biochemical compound that protects said W303-1B/Δydl120w::Kan^{R} from cellular stress.

5. The method of claim 4, wherein the cellular stress is caused by metal ions or pro-oxidant molecules.

6. The method of claim 5, wherein the metal ions are iron ions.

7. The method of claim 5, wherein the metal ions are copper ions.

8. The method of claim 4, wherein the cellular stress is caused by specific enzymatic reactions or the use of non-fermentable carbon sources in a culture media.

9. The method of claim 3, wherein said pharmaceutically active compound is used to treat human diseases caused by the pathological accumulation of iron or copper or by oxidative stress.

10-18. (canceled)

19. The method of claim 8, wherein the non-fermentable carbon sources in the culture media are selected from the group consisting of ethanol, glycerol, raffinose and lactate.

20. The method of claim 3, further comprising treating a human disease with the pharmaceutically active compound.
